(19) United States
(12) Patent Application Publication
(10) Pub. No.: US 2004/0242669 A1
(43) Pub. Date: Dec. 2, 2004

(54) Method of treating deficits associated with brain injury

(76) Inventor: Filippo Drago, Catania (IT)

Correspondence Address: GLAXOSMITHKLINE, Corporate Intellectual Property - UW2220, P.O. Box 1539, King of Prussia, PA, 19406-0939, US

(21) Appl. No.: 10/855,685

(22) Filed: May 27, 2004

Related U.S. Application Data
(60) Provisional application No. 60/473,590, filed on May 27, 2003.

(51) Int. Cl. A61K 31/404

(52) U.S. Cl. 514/418

(57) ABSTRACT

The present invention is directed to a method for treating the motor, behavioral, and cognitive deficits resulting from brain injury comprising administering a safe and effective amount of a dopamine agonist to a patient in need thereof.

### FIELD OF THE INVENTION

The present invention is directed to a method of treating motor, behavioral and cognitive deficits that result from brain injury, and especially to the use of ropinirole in such treatment.

### BACKGROUND OF THE INVENTION

Brain injury may occur when the brain is deprived of oxygen. Anoxic brain injuries occur when the brain receives no oxygen, such as with a near-drowning victim. Hypoxic brain injuries occur when the brain receives some, but not enough, oxygen, such as with a stroke victim. Anoxic and hypoxic brain injuries may results from airway obstruction, near-drowning, throat swelling, choking, strangulation, crush injuries to the chest, electrical shock, lightening strike, trauma to the head or neck, blood loss, shock, vascular disruption, heart attack, stroke, arteriovenous malformation, aneurysm, intracranial surgery, intracranial tumor, infectious disease, meningitis, metabolic disorders, hepatic encephalopathy, uremic encephalopathy, seizure disorders, lead poisoning, carbon monoxide poisoning, cardiac arrest, coronary artery bypass graft (CABG) surgery, and other conditions.

Both anoxic and hypoxic brain injuries often cause neurological abnormalities that may result in motor, behavioral, and cognitive deficits. Such deficits may result in significant handicap, disability and lost quality of life. Thus, there is a great need for a novel treatment for the motor, behavioral and cognitive deficits that result from brain injury.

### SUMMARY OF THE INVENTION

The present invention is directed to a method for treating the motor, behavioral, and cognitive deficits resulting from brain injury comprising administering a safe and effective amount of a dopamine agonist to a patient in need thereof.

### DETAILED DESCRIPTION OF THE INVENTION

The present invention is directed to a method for treating the motor, behavioral, and cognitive deficits resulting from brain injury comprising administering a safe and effective amount of a dopamine agonist to a patient in need thereof.

The motor, behavioral, and cognitive deficits that may result from anoxic and hypoxic brain injuries include speech disorders, language disorders, gait abnormalities, involuntary bodily movements, neglect, memory loss, disorientation, depression, paralysis and paresis.

The skilled artisan will appreciate that a patient who suffers a brain injury may experience motor deficits, behavioral deficits, or cognitive deficits, or such a patient may experience any combination of these deficits. Accordingly, in one aspect, the present invention is directed to a method for treating the motor deficits resulting from brain injury by administering a dopamine agonist to a patient in need thereof. In another aspect, the present invention is directed to a method for treating the behavioral deficits resulting from brain injury by administering a dopamine agonist to a patient in need thereof. In yet another aspect, the present invention is directed to a method for treating the cognitive deficits resulting from brain injury by administering a dopamine agonist to a patient in need thereof.

As used herein, “dopamine agonist” refers to compound that mimics the action of dopamine at a dopamine receptor. Currently, there are five known dopamine receptors. Dopamine agonists useful in the present invention include: apomorphine, bromocriptine, cabergoline, lisuride, pergolide, pramipexole, dihydroergocryptine (“DHECP”) and ropinirole which are all commercially available. Common daily doses and dosing regimens are known by those skilled in the art. In addition, several resources available to those skilled in the art, such as Goodman & Gilman's The Pharmacological Basis of Therapeutics, describe common doses and dosing regimens for these dopamine agonists.

Ropinirole is 4-[2-(di-n-propylamino)ethyl]-1,3-dihydro-2H-indolin-2-one hydrochloride (ropinirole). This compound has been found to be a potent CNS active non-ergot dopamine receptor agonist (U.S. Pat. Nos. 4,824,860 and 4,912,126), which may exhibit antihypertensive and anti-anginal properties (U.S. Pat. Nos. 4,452,808 and 4,588,740). The hydrochloride salt of ropinirole is approved for human use in therapy to treat Parkinson's disease and is sold in the US as REQUIP®. Processes for the production of ropinirole hydrochloride are disclosed in U.S. Pat. Nos. 4,997,954 and 5,336,781.

As used herein, “treatment” means the amelioration, alleviation, or prevention of one or more of the symptoms or effects associated with the deficiency being treated. The skilled artisan will appreciate that “prevention” is not an absolute term. In medicine, “prevention” is understood to refer to the prophylactic administration of a drug to substantially diminish the likelihood or severity of a condition, or a symptom or effect thereof, or to delay the onset of such condition, or symptom or effect thereof.

The dopamine agonists of the invention may also exhibit a protective effect against the motor, behavioral, and cognitive deficits resulting from brain injury when administered prior to the brain injury. Therefore, the dopamine agonists of the invention may also be administered prophylactically in a patient at risk of suffering from a brain injury, such as a patient at risk of suffering from a stroke.

As used herein, “safe and effective amount” means an amount of the dopamine agonist sufficient to significantly induce a positive modification in the deficiency to be treated but low enough to avoid serious side effects (at a reasonable benefit/risk ratio) within the scope of sound medical judgment. A safe and effective amount of a dopamine agonist useful the invention will vary with the particular compound chosen (e.g. consider the potency, efficacy, and half-life of the compound); the route of administration chosen; the condition being treated; the severity of the condition being treated; the age, size, weight, and physical condition of the patient being treated; the medical history of the patient to be treated; the duration of the treatment; the nature of concurrent therapy; the desired therapeutic effect; and like factors, but can nevertheless be routinely determined by the skilled artisan.

As used herein, “patient” refers to a human or other animal.

The dopamine agonists useful in the invention may be administered by any suitable route of administration, including both systemic administration and topical administration. Systemic administration includes oral administration, parenteral administration, transdermal administration, rectal administration, and administration by inhalation. Parenteral administration refers to routes of administration other than enteral, transdermal, or by inhalation, and is typically by injection or infusion. Parenteral administration includes intravenous, intramuscular, and subcutaneous injection or infusion. Inhalation refers to administration into the patient's lungs whether inhaled through the mouth or through the nasal passages. Topical administration includes application to the skin as well as intraocular, otic, intravaginal, and intranasal administration.

The dopamine agonists useful in the invention may be administered once or according to a dosing regimen wherein a number of doses are administered at varying intervals of time for a given period of time. For example, doses may be administered one, two, three, or four times per day. Doses may be administered until the desired therapeutic effect is achieved or indefinitely to maintain the desired therapeutic effect. Suitable dosing regimens for a compound of the invention depend on the pharmacokinetic properties of that compound, such as absorption, distribution, and half-life, which can be determined by the skilled artisan. In addition, suitable dosing regimens, including the duration such regimens are administered, for a compound of the invention depend on the condition being treated, the severity of the condition being treated, the age and physical condition of the patient being treated, the medical history of the patient to be treated, the nature of concurrent therapy, the desired therapeutic effect, and like factors within the knowledge and expertise of the skilled artisan. It will be further understood by such skilled artisans that suitable dosing regimens may require adjustment given an individual patient's response to the dosing regimen or over time as individual patient needs change.

Typical daily dosages may vary depending upon the particular route of administration chosen. The daily dosage required for an adult patient may, for example, be an oral dosage of between 0.1 mg and 100 mg, preferably between 0.5 mg and 25 mg; or an intravenous, subcutaneous or intramuscular dosage of between 0.1 mg and 25 mg, preferably between 0.1 mg and 15 mg, of the dopamine agonist. The dopamine agonist may be administered for a period of continuous therapy.

The dopamine agonists useful in the invention will normally, but not necessarily, be formulated into pharmaceutical compositions prior to administration to a patient. Such pharmaceutical compositions comprise a dopamine agonist useful in the invention and a pharmaceutically-acceptable excipient. Pharmaceutical compositions may be prepared and packaged in bulk form wherein a safe and effective amount of a dopamine agonist useful in the invention can be extracted and then given to the patient such as with powders or syrups. Alternatively, pharmaceutical compositions may be prepared and packaged in unit dosage form wherein each physically discrete unit contains a safe and effective amount of a dopamine agonist useful in the invention.

As used herein, “pharmaceutically-acceptable excipient” means a pharmaceutically acceptable material, composition or vehicle involved in giving form or consistency to the pharmaceutical composition. Each excipient must be compatible with the other ingredients of the pharmaceutical composition when commingled such that interactions which would substantially reduce the efficacy of the dopamine agonist useful in the invention when administered to a patient and interactions which would result in pharmaceutical compositions that are not pharmaceutically acceptable are avoided. In addition, each excipient must of course be of sufficiently high purity to render it pharmaceutically-acceptable.

The dopamine agonist useful in the invention and the pharmaceutically-acceptable excipient or excipients will typically be formulated into a dosage form adapted for administration to the patient by the desired route of administration. For example, dosage forms include those adapted for (1) oral administration such as tablets, capsules, caplets, pills, troches, powders, syrups, elixers, suspensions, solutions, emulsions, sachets, and cachets; (2) parenteral administration such as sterile solutions, suspensions, and powders for reconstitution; (3) transdermal administration such as transdermal patches; (4) rectal administration such as suppositories; (5) inhalation such as aerosols and solutions; and (6) topical administration such as creams, ointments, lotions, solutions, pastes, sprays, foams, and gels.

Suitable pharmaceutically-acceptable excipients will vary depending upon the particular dosage form chosen. In addition, suitable pharmaceutically-acceptable excipients may be chosen for a particular function that they may serve in the composition. For example, certain pharmaceutically-acceptable excipients may be chosen for their ability to facilitate the production of uniform dosage forms. Certain pharmaceutically-acceptable excipients may be chosen for their ability to facilitate the production of stable dosage forms. Certain pharmaceutically-acceptable excipients may be chosen for their ability to facilitate the carrying or transporting the compound or compounds of the invention once administered to the patient from one organ, or portion of the body, to another organ, or portion of the body. Certain pharmaceutically-acceptable excipients may be chosen for their ability to enhance patient compliance.

Suitable pharmaceutically-acceptable excipients include the following types of excipients: Diluents, fillers, binders, disintegrants, lubricants, glidants, granulating agents, coating agents, wetting agents, solvents, co-solvents, suspending agents, emulsifiers, sweetners, flavoring agents, flavor masking agents, coloring agents, anticaking agents, hemectants, chelating agents, plasticizers, viscosity increasing agents, antioxidants, preservatives, stabilizers, surfactants, and buffering agents. The skilled artisan will appreciate that certain pharmaceutically-acceptable excipients may serve more than one function and may serve alternative functions depending on how much of the excipient is present in the formulation and what other ingredients are present in the formulation.

Skilled artisans possess the knowledge and skill in the art to enable them to select suitable pharmaceutically-acceptable excipients in appropriate amounts for use in the invention. In addition, there are a number of resources that are available to the skilled artisan which describe pharmaceutically-acceptable excipients and may be useful in selecting suitable pharmaceutically-acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The pharmaceutical compositions of the invention are prepared using techniques and methods known to those skilled in the art. Some of the methods commonly used in the art are described in Reminqton's Pharmaceutical Sciences (Mack Publishing Company).

Ropinirole

In one embodiment, the present invention is directed to a method for treating the motor, behavioral, and cognitive deficits resulting from brain injury comprising administering a safe and effective amount of ropinirole or a pharmaceutically-acceptable salt or solvate thereof to a patient in need thereof. In another embodiment, the present invention is directed to a method for treating the motor deficits resulting from brain injury comprising administering a safe and effective amount of ropinirole or a pharmaceutically-acceptable salt or solvate thereof to a patient in need thereof.

Each dosage unit of ropinirole for oral administration may comprise from 0.1 to 50 mg of ropinirole; preferably 0.25-10 mg. For parenteral administration, each dosage unit may comprise from 0.1 to 15 mg of ropinirole. By way of example, a representative regimen for the administration of ropinirole is 0.25-5 mg of ropinirole 2 or 3 times a day.

In one embodiment, the dose of ropinirole administered to a patient is titrated such that an optimal dose for that patient is identified. A representative titration schedule is as follows: Patients are initially treated with ropinirole at the low end of the recommended dose, for example a dose of about 1 mg once per day. An example of a typical dosing regimen may involve increasing the amount of ropinirole gradually on a weekly basis until the patient exhibits a therapeutic effect or intolerance. Table A details 2 suitable examples of such a dosing regimen. Alternatively, if desired, a more rapid dosing regimen may also be used.

TABLE A

| | |
|---|---|
| An Example of a Standard | An Example of a Rapid |
| Dosing Regimen | Dosing Regimen |

| | Dose (mg once | | Dose (mg once |
|---|---|---|---|
| Week | per day) | Week | per day) |
| 1 | 1 | 1 | 2 |
| 2 | 2 | 2 | 4 |
| 3 | 3 | 3 | 6 |
| 4 | 4 | 4 | 8 |
| 5 | 6 | 5 | 12 |
| 6 | 8 | 6 | 16 |
| 7 | 10 | 7 | 20 |
| 8 | 12 | 8 | 24 |
| 9 | 14 | 9 | 28 |
| 10 | 16 | 10 | 32 |
| 11 | 20 | 11 | 36 |
| 12 | 24 | 12 | 40 |
| 13 | 28 | | |
| 14 | 32 | | |
| 15 | 36 | | |
| 16 | 40 | | |

In general, the, dosage of ropinirole should be increased gradually from a starting, dose of about 1-2 mg of ropinirole per day and then increased every 1-7 days to a, maximum dose of per day of about 30.0 mg of ropinirole per day. Providing patients do not experience intolerable side effects, the dosage should be titrated to achieve a maximal therapeutic effect. The effective dose of ropinirole is usually between about 1 mg per day to about 50 mg per day. More usually, the effective dose is between about 3 mg and about 30 mg per day.

Ropinirole or a pharmaceutically acceptable salt or solvate thereof may be formulated for administration by any route, and examples are oral, sub-lingual, transdermal, rectal, topical, parenteral, intravenous or intramuscular administration. Preparations may, if desired, be designed to give either immediate or slow release of the ropinirole or a pharmaceutically acceptable salt or solvate thereof.

In one embodiment, the ropinirole or a pharmaceutically-acceptable salt or solvate thereof is provided in the form of a transdermal patch. Suitable patch formulations for transdermally administering ropinirole hydrochloride include those described in U.S. Pat. No. 5,807,570.

In another embodiment, ropinirole or a pharmaceutically-acceptable salt or solvate thereof is administered as a controlled release or delayed release formulation. By controlled release is meant any formulation technique wherein release of the active substance from the dosage from is modified to occur at a slower rate than that from an immediate release product, such as a conventional oral tablet or capsule. By delayed release is meant any formulation technique wherein release of the active substance from the dosage form is modified to occur at a later time than that from a conventional immediate release product. The subsequent release of active substance from a delayed release formulation may also be controlled as defined above. Examples of controlled release formulations which are suitable for incorporating ropinirole or a pharmaceutically-acceptable salt or solvate thereof are described in International Patent Application WO 01/78688 and the following references:

Sustained Release Medications, Chemical Technology Review No.177. Ed.

J. C. Johnson. Noyes Data Corporation 1980.

Controlled Drug Delivery, Fundamentals and Applications, 2nd Edition. Eds.

J. R. Robinson, V. H. L. Lee. Mercel Dekkes Inc. New York 1987.

Examples of delayed release formulations which are suitable for incorporating ropinirole or a pharmaceutically-acceptable salt or solvate thereof are described in:

Remington's Pharmaceutical Sciences 16th Edition, Mack Publishing Company 1980, Ed. A. Osol.

### Experimental

1. Animals

Male rats of the Sprague-Dawley strain (purchased from Charles River, Italy) weighing 280±20 g were used throughout all experiments. The animals were housed two-three to a cage under a constant light-dark cycle (lights on between 8.00 and 20.00) at 21° C. Commercial food and tap water were available ad libitum. All animals were used only once in the behavioral experiments.

2. Experimental Ischemia

A number of animals were subjected to a manipulation of the four major arteries of the brain with a method similar to that described by Pulsinelli et al., Ann. Neurol, 11, 491-498 (1982). Both vertebral arteries were cauterised under sodium pentobarbital anaesthesia and polyethylene cuffs PE-10 were placed loosely around the common carotid arteries without completely interrupting carotid blood flow. Two months after operation, all surviving animals showing no neurological gross abnormalities were admitted to drug treatment. Animals undergoing a sham operation were considered as controls.

Another group of animals were implanted under ether anaesthesia with a permanent plastic cannula in the right lateral ventricle (foramen interventriculare, Konig and Klippel A 6360) with another cannula into the right jugular vein. These animals were sacrificed at the end of behavioral procedure and the brains were utilized for biochemical analysis. Data were taken from animals showing no abnormalities at the postmortem examination. All experiments were carried out according to the European Community Council Directive 86/609/ECC and efforts were made to minimize animal suffering and to reduce the number of animals used.

3. Drugs and Treatment

Ropinirole (ropinirole hydrochloride, SmithKline Beecham, Italy) and dihydroergocryptine (DHECP) α-dihydroergocryptine methanesulfonate, Monsanto, Italy) were dissolved in saline and injected subcutaneously (s.c.) at the dose of 0.5 and 1 mg/kg/day for 7 days. In the experiments with kainate-induced convulsions, ropinirole or DHECP were injected intravenously (i.v.) 30 min prior, and again at the same time of the intracerebroventricular (i.c.v) injection on kainic acid; Control animals received an injection of saline alone with the same procedure.

Kainic acid (Sigma, USA) was dissolved in saline and injected i.c.v. at the dose of 10 μg/2 μi.

4. Behavioral Tests

Shuttle-box active avoidance acquisition was studied in a single session test as described in Bohus and De Wied, Endogenous Peptides and Learning and Memory Processes. Academic Press, New York, pp59-775, (1981). Briefly, the rats were trained to avoid the unconditioned stimulus (US) of a scrambled electrical foot-shock (0.20 mA) delivered through the grid floor. The conditioned stimulus (CS) was a buzzer presented for 3 s prior to the US. If no escape occurred within 20 s of CS/US presentation, the shock was terminated. A maximum of 30 conditioning trials were given with a variable intertrial interval averaging 60 s. The learning criterion was five consecutive conditioned avoidance responses (CARs). For those animals that reached the criterion in less than 30 trials, the remaining trials until 30 were considered as CARs. Indexes of avoidance behaviour were the total number of CARs and the percent number of learners per group.

Passive avoidance behaviour was studied in a step-through type of passive avoidance situation (Ader et al., Psycon Sci, 26, 125-128 (1972)). Briefly the rats were adapted to the apparatus consisting of a large dark compartment equipped with a grid floor and a mesh-covered elevated runway attached to the front centre of the dark chamber. Adaptation training was followed by a single trial in which the rats were placed on the elevated platform and allowed to enter the dark box. Three such trials were given on the next day with an intertrial interval of 5 min after the trial. The rats received a single 2-s unavoidable scrambled foot-shock (0.20 mA) immediately after entering the dark compartment. Retention of the response was tested 24 h after the learning trial. The rats were placed on the elevated runway and the latency to re-enter the shock compartment was recorded up to a maximum of 300 s.

Spontaneous motor activity was scored in a circular open field arena as described by Weijnen and Slangen, Pituitary, Adrenal and the Brain, pp221-235 (1982). Animals were put inside a circular lit-up arena, with the bottom divided into 27 areas of equal size. The behaviour of each animal was observed for a period of 5 min and the occurrence of these items was recorded: ambulation (number of areas explored with at least the forelegs); rearing (number of episodes in which the animal raises on rear legs with a muzzle towards the centre or towards one of the walls of the field); grooming (number of episodes in which the animal licks its fur, legs or genitals); defecation (number of fecal boluses released during the observation).

5. Experimental Convulsions

Over a 30-min observation, the following items were recorded per each animal receiving an i.c.v. injection of kainic acid: latency in s to the first tonic-clonic seizure; total number of seizures, percent rate of mortality (checked 2 h after the administration of the convulsive agent).

6. Biochemical Analysis

At the end of the behavioral studies, the brain of animals sacrificed by decapitation were taken and rapidly frozen for analysis. Dissection and weighing were carried out at −25° C. The frozen forebrains were immediately powered under liquid nitrogen by an electromechanical apparatus and stored at 80° C. The assay of reduced and oxidized glutathione by 5,5′-dithibis-(2-nitrobenzoic acid) in frozen forebrain powder was carried out within 3 h, utilizing perchloric acid and N-ethylmaleimide in presence of ethylenediamidetetraacetic acid for the extraction procedure (Tietze, Anal Biochem, 27, 502-522, (1969)).

7. Experimental Design

In the first experiment, the effects of ropinirole or DHECP were studied on amnesia induced by hypobaric hypoxia in rats. After a 7-day pretreatment with ropinirole, DHECP or saline, a group of animals was subjected to acute hypobaric hypoxia by means of a hypobaric chamber (Chantiers et Ateliers de Bretagne, France) causing depression to 300 mmHg (Boismare et al., Gerentology, 24, 6-13 (1978)). After 3 min in the chamber, the animals were removed and admitted for behavioral testing. Intact control rats were also introduced into the chamber but not subjected to hypobaric hypoxia.

Animals with 2-month chronic brain occlusive ischemia, were subjected to a treatment with ropinirole, DHECP or saline for 7 days. At the end of treatment, the animals were admitted to behavioral testing. Controls were those animals with sham manipulation of brain arteries.

Behavioral scoring of kainate-induced convulsions was started immediately after i.c.v. injection of the drug and proceeded for 30 min. Ropinirole, DHECP or saline were injected twice, 30 min prior and, again, at the same time of the i.c.v. administration of kainic acid.

All animals used for hypobaric hypoxia induced amnesia and for chronic brain occlusive ischemia was sacrificed at the end behavioral procedure for biochemical analyses.

8. Statistical Analysis

The two-way ANOVA and the posthoc Dunnett's test for multiple comparisons were used for statistical analysis of parametric data. The Mann-Whitney U-test was used for non-parametric data and the Fisher exact t-test for frequencies. A p level of 0.05 or less was considered as indicative of significant difference.

### EXAMPLES

### Example 1

### Effects of Ropinirole or Dihydroergocryptine (DHECP) on Hypobaric Hypoxia-Induced Amnesia

Table 1 shows the results concerning the influence of ropinirole or DHECP treatment on behavioral performance of animals with hypobaric hypoxia-induced amnesia. A decrease in learning and memory capacity in these animals was indicated by the reduction in the number of CARs and the percent number of learners in the shuttle-box and in the latency to re-enter the dark box. The treatment with ropinirole or DHECP induced an increase in these behavioral parameters, however, for the latter only with the greater dose.

No major changes were found in spontaneous behaviour scored in the open field test after the application of hypobaric hypoxia (Table 1). However, ambulation and rearing of animals treated with ropinirole or DHECP was higher than those of intact controls and animals subjected to hypobaric hypoxia and treated with saline. No difference was found between 0.5 and 1 mg/kg doses for the two drugs in this respect.

TABLE 1
Effects of saline (SAL), ropinirole (ROP) or dihydroergocryptine (DHECP)
on behavioral performance of animals with hypobaric hypoxia-induced amnesia

| Behavioral | | | ROP | ROP | DHECP | DHECP |
|---|---|---|---|---|---|---|
| parameters | INTACT | SAL | 0.5 mg/kg | 1 mg/kg | 0.5 mg/kg | 1 mg/kg |
| CARs | 18.4 ± 1.2 | 13.0 ± 1.4^{a} | 16.8 ± 1.3^{b} | 17.2 ± 1.5^{b} | 13.0 ± 1.3^{a} | 18.1 ± 1.2^{b} |
| Learners | 90 | 30^{c} | 60^{d} | 70^{d} | 30^{c} | 80^{d} |
| Latency | 78 | 21^{e} | 47^{e,f} | 65^{f} | 30^{e} | 74^{f} |
| Ambulation | 56.8 ± 5.9 | 49.5 ± 5.4^{a} | 67.4 ± 5.4^{b} | 68.3 ± 4.5^{b} | 68.9 ± 6.6^{b} | 73.1 ± 5.6^{a,b} |
| Rearing | 13.0 ± 1.0 | 11.8 ± 1.4^{a} | 16.7 ± 1.5^{a,b} | 17.5 ± 1.2^{a,b} | 16.4 ± 1.3^{a,b} | 16.5 ± 1.9^{b} |
| Grooming | 24.6 ± 3.0 | 27.4 ± 3.9 | 21.7 ± 3.3 | 23.7 ± 2.1 | 22.2 ± 2.3 | 23.3 ± 2.1 |
| Defecation | 5.4 ± 1.0 | 4.7 ± 0.4 | 3.1 ± 0.8 | 2.3 ± 0.9^{a} | 3.4 ± 0.9 | 3.7 ± 1.3 |
| Intact animals were rats not subjected to hypobaric hypoxia. Animals were 20 per each group. Ropinirole or DHECP were injected s.c. at the dose of 0.5 or 1 mg/kg/day for 7 days. CARs, Ambulation, Rearing. Grooming and Defecation items | | | | | | |
| #are expressed as means ± S.E.M. Learners item is expressed in percent. Latency item is expressed in median. | | | | | | |

^{a}Significant difference vs. intact controls (P < 0.05, Dunnett's t-test for multiple comparisons)
^{b}Significant difference vs. saline-treated controls (P < 0.05, Dunnett's t-test for multiple comparisons)
^{c}Significant difference vs. intact controls (P < 0.05, Fisher test).
^{d}Significant difference vs. saline treated controls (P < 0.05, Fisher test).
^{e}Significant difference vs. intact controls (P < 0.05, Mann-Whitney U test).
^{f}Significant difference vs. saline-treated controls (P < 0.05, Mann-Whitney U test).

The above results in Table 1 demonstrate that pre-treatment with ropinirole or DHECP increased learning and memory behaviours in animals subjected to hypobaric hypoxia, a model of global ischemia. This finding implies that ropinirole or DHECP is likely to be useful in prophylactic treatment of subjects undergoing surgical procedures, e.g. coronary artery bypass graft (CABG) surgery, which might generate cognitive deficits.

### Example 2

### Effects of Ropinirole or Dihydroergocryptine (DHECP) on Chronic Brain Occlusive Ischemia

Saline-injected rats with chronic brain occlusive ischemia showed a reduction in cognitive parameters scored with the active and passive avoidance tests (Table 2). No statistically significant change was found in the same parameters after treatment with ropinirole or DHECP, except for the latency to re-enter the dark-box that appeared to be increased after drug treatment. Animals with chronic brain occlusive ischemia showed a decrease in spontaneous motor activity (ambulation and rearing) in the open field test. Ropinirole or DHECP treatment was followed by increased ambulation and rearing, to a level similar to that of intact controls. No other effects of drug treatment were found in open field spontaneous behaviour of animals with chronic brain occlusive ischemia.

TABLE 2
Effects of saline (SAL), ropinirole (ROP) or dihydroergocryptine (DHECP)
on behavioral performance on animals with chronic brain occlusive ischemia

| Behavioral | | | ROP | ROP | DHECP | DHECP |
|---|---|---|---|---|---|---|
| parameters | INTACT | SAL | 0.5 mg/kg | 1 mg/kg | 0.5 mg/kg | 1 mg/kg |
| CARs | 17.4 ± 1.1 | 9.5 ± 0.8^{a} | 10.8 ± 0.3^{a} | 10.2 ± 1.5^{a} | 10.6 ± 1.1^{a} | 14.1 ± 1.1^{a} |
| Learners | 80 | 20^{b} | 10^{b} | 10^{b} | 20^{b} | 10^{b} |
| Latency | 70 | 21^{c} | 43^{c,d} | 55^{d} | 46^{c,d} | 58^{d} |
| Ambulation | 50.1 ± 5.2 | 49.5 ± 5.4 | 67.4 ± 5.4^{a,e} | 68.3 ± 4.5^{a,e} | 68.9 ± 6.6^{a,e} | 73.1 ± 5.6^{a,e} |
| Rearing | 13.0 ± 1.0 | 11.8 ± 1.4 | 16.7 ± 1.5^{e} | 17.5 ± 1.2^{e} | 16.4 ± 1.3^{e} | 16.5 ± 1.9^{e} |
| Grooming | 24.6 ± 3.0 | 27.4 ± 3.9 | 21.7 ± 3.3 | 23.7 ± 2.1 | 22.2 ± 2.3 | 23.3 ± 2.1 |
| Defecation | 5.4 ± 1.0 | 4.7 ± 0.4 | 3.1 ± 0.8^{a} | 2.3 ± 0.9^{a} | 3.4 ± 0.9^{a} | 3.7 ± 1.3^{a} |
| Intact animals were rats not subjected to chronic brain occlusive ischemia. Animals were 20 per each group. Ropinirole or DHECP were injected s.c. at the dose of 0.5 or 1 mg/kg/day for 7 days. CARs, Ambulation, Rearing, Grooming and Defecation | | | | | | |
| #items are expressed as means ± S.E.M. Learners item is expressed in percent. Latency item is expressed in median. | | | | | | |

^{a}Significant difference vs. intact controls (P < 0.05, Dunnett's t-test for multiple comparisons)
^{b}Significant difference vs. intact controls (P < 0.05, Fisher test)
^{c}Significant difference vs. intact controls (P < 0.05, Mann-Whitney U test).
^{d}Significant difference vs. saline-treated controls (P < 0.05, Mann-Whitney U test).
^{e}Significant difference vs. saline-treated controls (P < 0.05, Dunnett's t-test for multiple comparisons)

The above results in Table 2 demonstrate that administration of ropinirole or DHECP 2 months after the original insult was effective in enhancing motor function over saline-injected controls. This finding implies that ropinirole or DHECP is likely to be useful in enhancing recovery of patients who have suffered neuronal injury, e.g., stroke and traumatic brain injury, even if significant time has elapsed since the primary insult.

### Example 3

### Effects of Ropinirole or Dihydroergocryptine (DHECP) on Kainate-Induced Epilepsy

The treatment with ropinirole or DHECP did not modify the latency to the first tonic-clonic seizure nor the total number of kainate-induced seizures (Table 3). However, a significant decrease in percent mortality rate was observed in animals treated with either drug.

TABLE 3
Effects of saline (SAL), ropinirole (ROP) or
dihydroergocryptine (DHECP) on kainate-induced epilepsy

| Behavioral | | ROP | ROP | DHECP | DHECP |
|---|---|---|---|---|---|
| parameters | SAL | 0.5 mg/kg | 1 mg/kg | 0.5 mg/kg | 1 mg/kg |
| Latency (s) | 58.5 ± 7.8 | 69.4 ± 8.9 | 65.4 ± 6.9 | 56.9 ± 8.5 | 69.2 ± 6.5 |
| Seizures | 5.6 ± 1.2 | 7.1 ± 0.9 | 6.9 ± 0.9 | 7.8 ± 1.0 | 5.7 ± 1.1 |
| Mortality | 60 | 20^{a} | 10^{a} | 10^{a} | 10^{a} |
| (%) | | | | | |
| Animals were injected i.c.v. with kainic acid (10 μg/2 μl). Over 30-min observation, the following items were recorded: latency (s) to the first tonic-clonic seizure; total number of seizures; percent mortality. Saline, ropinirole or DHECP were injected | | | | | |
| #s.c. twice, 30 min prior and, again, at the same time of the i.c.v. administration of kainic acid. Animals were 20 per each group. Latency and seizures items are expressed in mean ± S.E.M. | | | | | |

^{a}Significant difference vs. saline-treated controls (P < 0.05, Fisher t-test).

The above results in Table 3 demonstrate that ropinirole or DHECP may be effective in ameliorating the effects of pharmacologically induced brain injury.

### Example 4

### Effects of Ropinirole or Dihydroergocryptine on Glutathione Content

In all brain areas examined, a decrease in reduced glutathione content (and in glutathione redox index) was found after hypobaric hypoxia or chronic brain occlusive ischemia. In animals with hypobaric hypoxia, an increase of these parameters was found with both doses of ropinirole or DHECP in the cortex, hippocampus and hypothalamus and only with the greater dose of the drugs in the striatum (Table 4).

The same was found in animals with chronic brain occlusive ischemia, but the decrease of reduced glutathione content and of glutathione redox index in the striatum was not effected by the drug treatment (Table 5.)

TABLE 4
Effects of saline (SAL), ropinirole (ROP) or dihydroergocryptine (DHECP)
on the reduced glutathione content (in mM) and on glutathione redox index
(glutathione reduced/glutathione oxidized ration) in various brain areas of rats
subjected to hypobaric hypoxia (hypoxia)

| | Brain Areas | | | |
|---|---|---|---|---|
| Experimental groups | Cortex | Striatum | Hippocampus | Hypothalamus |
| Intact Controls | 2.4 ± 0.1 (2.1) | 2.6 ± 0.1 (2.8) | 2.4 ± 0.1 (2.6) | 2.7 ± 0.2 (2.8) |

Hypobaric hypoxia

| | | | | |
|---|---|---|---|---|
| +SAL | 1.2 ± 0.1^{a} (0.5)^{b} | 1.8 ± 0.1^{a} (0.9)^{b} | 1.6 ± 0.1^{a} (0.4)^{b} | 1.6 ± 0.1^{a} (0.3)^{b} |
| +ROP 0.5 mg/kg | 2.3 ± 0.1^{c} (2.6)^{d} | 1.7 ± 0.1^{a} (0.8)^{b} | 2.0 ± 0.1^{b} (1.9)^{c} | 2.9 ± 0.2^{b} (2.4)^{c} |
| +ROP 1 mg/kg | 2.4 ± 0.1^{c} (2.2)^{d} | 2.6 ± 0.1^{c} (1.8)^{d} | 2.2 ± 0.1^{c} (2.1)^{d} | 2.6 ± 0.2^{c} (2.6)^{d} |
| +DHECP 0.5 mg/kg | 2.2 ± 0.2^{c} (2.5)^{d} | 1.8 ± 0.2^{a} (0.7)^{b} | 2.1 ± 0.2^{c} (1.8)^{d} | 2.8 ± 0.2^{c} (2.6)^{d} |
| +DHECP 1 mg/kg | 2.5 ± 0.2^{c} (2.3)^{d} | 2.5 ± 0.2^{c} (1.7)^{d} | 2.4 ± 0.2^{c} (2.2)^{d} | 2.8 ± 0.2^{c} (2.8)^{d} |

Values are mean ± S.E.M. In parentheses is indicated the glutathione redox index (glutathione reduced/glutathione oxidized ratio). Animals were 20 per each group.
^{a}Significant difference vs. intact controls (P < 0.05, Dunnett's test)
^{b}Significant difference vs. intact controls (P < 0.05, Fisher t test)
^{c}Significant difference vs. intact controls (P < 0.05, Dunnett's test)
^{d}Significant difference vs. intact controls (P < 0.05, Fisher t test)

TABLE 5
Effects of saline (SAL), ropinirole (ROP) or dihydroergocryptine (DHECP)
on the reduced glutathione content (in mM) and on the glutathione redox index
(glutathione reduced/glutathione oxidized ratio) in various brain areas of rats
subjected to chronic brain occlusive ischemia (ISCH)

| | Brain Areas | | | |
|---|---|---|---|---|
| Experimental groups | Cortex | Striatum | Hippocampus | Hypothalamus |
| Intact Controls | 2.4 ± 0.1 (2.1) | 2.6 ± 0.1 (2.8) | 2.4 ± 0.1 (2.6) | 2.7 ± 0.2 (2.8) |

Ischemia

| | | | | |
|---|---|---|---|---|
| +SAL | 1.6 ± 0.1^{a} (1.0)^{b} | 1.6 ± 0.1^{a} (1.6)^{b} | 1.4 ± 0.1^{a} (0.8)^{b} | 1.8 ± 0.1^{a} (0.5)^{b} |
| +ROP 0.5 mg/kg | 2.2 ± 0.2^{c} (2.6)^{d} | 1.7 ± 0.1^{a} (0.8)^{b} | 2.1 ± 0.2^{c} (1.9)^{d} | 2.8 ± 0.2^{c} (2.3)^{d} |
| +ROP 1 mg/kg | 2.3 ± 0.1^{c} (2.2)^{d} | 1.6 ± 0.1^{a} (0.8)^{b} | 2.3 ± 0.2^{c} (2.1)^{d} | 2.7 ± 0.2^{c} (2.5)^{d} |
| +DHECP 0.5 mg/kg | 2.3 ± 0.1^{c} (2.5)^{d} | 1.8 ± 0.2^{a} (0.7)^{b} | 2.0 ± 0.2^{c} (1.8)^{d} | 2.8 ± 0.2^{c} (2.6)^{d} |
| +DHECP 1 mg/kg | 2.6 ± 0.2^{c} (2.3)^{d} | 1.7 ± 0.2^{a} (0.7)^{b} | 2.3 ± 0.3^{c} (2.2)^{d} | 2.7 ± 0.2^{c} (2.7)^{d} |

Values are mean ± S.E.M. In parentheses is indicated the glutathione redox index (glutathione reduced/glutathione oxidized ratio). Animals were 20 per each group.
^{a}Significant difference vs. intact controls (P < 0.05, Dunnett's test)
^{b}Significant difference vs. intact controls (P < 0.05, Fisher t test)
^{c}Significant difference vs. intact controls (P < 0.05, Dunnett's test)
^{d}Significant difference vs. intact controls (P < 0.05, Fisher t test)

The above results in Tables 4 and 5 demonstrate that ropinirole or DHECP appears to enhance the brain's anti-oxidative capacity. This has been postulated as an alternative mechanism of action for restoration of cerebral function after ischemic insult.

What is claimed is:

1. A method for treating the motor, behavioral, and cognitive deficits resulting from brain injury comprising administering a safe and effective amount of a dopamine agonist to a patient in need thereof.

2. A method for treating the motor deficits resulting from brain injury comprising administering a safe and effective amount of a dopamine agonist to a patient in need thereof.

3. The method according to claim 1 wherein the dopamine agonist is ropinirole or a pharmaceutically acceptable salt or solvate thereof.

4. The method as claimed in claim 3, wherein the pharmaceutically acceptable salt is the crystalline hydrochloride.

5. The method as claimed in claim 3 wherein ropinirole is administered orally.

6. The method as claimed in claim 3 wherein ropinirole is administered in an oral controlled release formulation.

7. The method as claimed in claim 3 wherein ropinirole is administered transdermally.

8. The method as claimed in claim 7 wherein ropinirole is administered as a patch formulation.
